(19) United States
(12) Patent Application Publication
(10) Pub. No.: US 2013/0291883 A1
(43) Pub. Date: Nov. 7, 2013

(54) Sol-Gel Compositions and Cosmetic Methods of Use

(71) Applicants: Allwyn COLACO, Morristown, NJ (US); Ashley L. HOWELL, Oakland, NJ (US)

(72) Inventors: Allwyn COLACO, Morristown, NJ (US); Ashley L. HOWELL, Oakland, NJ (US)

(73) Assignee: AVON PRODUCTS, INC., New York, NY (US)

(21) Appl. No.: 13/463,154

(22) Filed: May 3, 2012

(51) Int. Cl. A61K 8/73; A61Q 5/06; A45D 7/04; A61K 8/02

(52) U.S. Cl. 132/203; 424/70.13; 424/401

(57) ABSTRACT

Methods and cosmetic compositions are disclosed for applying a desired shape to keratin fibers, preventing or reducing frizzy appearance of hair, improving color retention of artificially colored hair, and improving the aesthetic appearance of hair. Methods for using cosmetic compositions according to the invention are silica sol-gel compositions comprising a homogeneous dispersion of (a) epoxysilane-functionalized silica particles; (b) a cosmetic particulate such as PTFE; and (c) a polysaccharide, such as chitosan.

### FIELD OF THE INVENTION

The present invention relates to methods and personal care/cosmetic compositions for application to a human integument. In particular, the methods and compositions are adapted to applying a desired shape to keratin fibers, preventing or reducing frizzy appearance of hair, improving color retention of artificially colored hair, and improving the aesthetic appearance of hair. More specifically, the invention relates to sol-gel compositions for applying to human integuments, such as hair, and in particular to methods utilizing said sol-gel compositions for shaping of keratin fibers, preventing or reducing frizzy appearance of hair, improving color retention of artificially colored hair, and improving the aesthetic appearance of hair, optionally without the use of heat.

### BACKGROUND OF THE INVENTION

The cross-linking of sot particles via a transition from an initial solution into a jellified state is called sol-gel processing. This route for silica fabrication is based on the tendency of silicic acids in solution to take part in condensation reactions, leading to a colloid with a continuous liquid phase. Upon application to a substrate followed by drying, such compositions form coatings. Sol-gel coatings made from modified silica sols containing various additives have been known in the surface material arts to be useful as binder materials, non-abrasive coatings, corrosion protection, spectacle refractive coatings, and textile coatings. Such coatings were typically applied to substrates such as glass, ceramic, wood, metal, stone, plastic, and concrete, and required a high-heat curing step, see, for example, Mahltig, Boris et al., Functionalisation of Textiles by Inorganic Sol-Gel Coatings, J. Mater, Chem. 15:4385-4398 (2005).

U.S. Pat. No. 7,247,350 B2 describes solvent-poor sol-gel systems for application to glass, ceramic, wood, metal, stone, plastic, and concrete substrates. Drying and hardening of the sol-gel system is described and drying in a forced-air oven is preferred.

PCT publication WO 03/014232 A1 describes materials for producing abrasion-proof, hydrophobic and/or oleoph.obic coatings which are characterized by containing at least one alkoxysilane having at least one polyfluoroalkyl and/or perfluoroalkyl group having a chain length of at least 20 carbon atoms. The coatings may be used for coating a substrate such as glass, ceramic, fabric, fiber, leather, wood, metal, plastic, paper, cardboard or a natural material.

Chapter 3, “Entrapment of Biopolymers into Sol-Gel Derived Silica Nanocomposites,” in Bio-inorganic Hybrid Nanomaterials (Wiley 2008) reviews sol-gel processing, including a section disclosing the use of various biopolymers (including chitosan) in sol-gel processing.

DE 10 2007 017 303 A1, filed Apr. 11, 2007, titled “Antibacterial Agent and its Use,” discloses permanent antibacterial finishes for fibers and textiles, and is hereby incorporated by reference in its entirety.

While alkoxysilane coating materials have been described in other technical fields, there remains a need for cosmetic compositions and methods, including hair care straightening compositions and methods, which are effective as coatings without the need for the application of heat, and that are appropriate and safe as cosmetic compositions. The coating materials in other technical fields were not suitable for forming a film on hair due to components that are undesirable for cosmetics, and keratin fibers or hair were not contemplated as a substrate. Keratin fibers would be unsuitable as a substrate under conditions previously utilized in the art.

Therefore, the invention addresses a need in the art to provide cosmetic compositions for methods of application to hair (keratin) fibers which provide straightening to curly hair. It is a further object of the invention to provide methods of using such cosmetic compositions for decreasing frizz of curly hair in the presence of humidity. It is also an object of the invention to provide methods utilizing such cosmetic compositions for straightening hair, decreasing frizz of curly hair in the presence of humidity, and protecting color treatment of hair.

### SUMMARY OF THE INVENTION

In accordance with the foregoing objectives and others, the present invention provides sol-gel compositions and methods for forming films on a surface, preferably a biological integument, and more preferably a keratin fiber (hair), where hair is given a desired shape (e.g., curly hair is straightened and/or straight hair is curled), hair shows decreased frizz in the presence of humidity, and artificially colored hair is able to retain its color longer. These effects may persist after at least one, two, or more washings and/or shampooings, and may not require the application of heat to achieve the desired effect.

Surprisingly, it has been found that a sol-gel composition comprising a dispersion (typically aqueous) of (a) organosilane-functionalized (typically epoxysilane-functionalized) inorganic oxide particles, combined with (b) a cosmetic particulate, and (c) at least one biopolymer, such as chitosan and/or chitosan derivatives, has excellent haircare properties, including straightening, holding a style (e.g., curl), reducing frizz under humid conditions (e.g., reducing frizz of straightened hair), and/or protecting color treatment.

In one aspect of the invention, the silica sol-gel composition comprises a dispersion (typically aqueous) of (a) organositane-functionalized (typically epoxysilane-functionalized) silica particles, combined with (b) a polytetrafluoroethylene (PTFE) cosmetic particulate, and (c) chitosan and/or a chitosan derivative. Durable films may be formed from these compositions without the need of elevated temperatures.

In one aspect of the invention, sol-gel compositions are provided comprising a dispersion of

(a) epoxy-functionalized inorganic oxide particles;

(b) a cosmetic particulate selected from the group consisting of inorganic particulates, organic polymeric particulates, pigments, lakes, silicon-based particulates, and combinations thereof; and

(c) a polysaccharide, such as chitosan or a chitosan derivative.

The inorganic oxide particles may be selected from silica, alumina, zinc oxide, and titanium dioxide particles, and mixtures thereof. The epoxy-functionalized inorganic oxide particles may be the reaction product of silica particles and at least one epoxysilane compound. Other inorganic oxide particles, such as silica, alumina, zinc oxide, and titanium dioxide, are suitable for use in forming epoxy-functionalized inorganic oxide particles for use in the invention in addition to silica particles, but epoxysitane-modified silica particles are particularly preferred. Although silica particles are a preferred embodiment, one of skill would understand that any particle which is capable of being functionatized by epoxy groups may be used.

In another aspect of the invention, epoxy-functionalized inorganic oxide particles may be the reaction product of silica particles and an epoxysilane of formula (I)

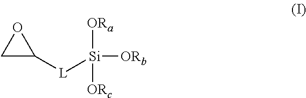

wherein Rₐ, R_{b}, and R_{c} are each independently C₁-C₆ alkyl, and are preferably all either ethyl or methyl;

L is a group of the form —X₁—(CR*₂)ₙ—X₂—(CR*₂)ₘ—X₃—;

wherein X₁, X₂, and X₃ are independently at each occurrence a bond (i.e X is absent), —O—, —NR^{N}—, —S—, —(OCH₂CH₂)_{y}—, or —(CH₂CH₂O)_{z}—, wherein y and z are independently an integer from 1 to 10, and n and m are independently an integer from 0 to 10 (preferably from 1 to 3);

R^{N} is independently at each occurrence hydrogen, lower alkyl (i.e., C₁-C₆ alkyl), phenyl, benzyl, or the like, and is most typically methyl; and

R* are independently at each occurrence C₁-C₄ alkyl, H, or halogen; and cosmetically suitable salts and derivatives thereof.

In one aspect of the invention, compositions for applying to a keratin fiber are provided comprising: a sot-gel composition comprising a dispersion of (a) epoxysilane-functionalized silica particles, wherein said epoxysilane-functionalized silica particles are the reaction product of silica particles with an epoxysilane of formula (Ia):

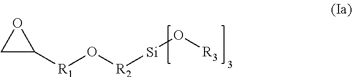

wherein R₁ and R₂ are each independently C₁-C₁₀ alkyl groups, and R₃ is a C₁-C₆ alkyl group, (b) polymeric particulates formed from the polymerization of fluorinated olefin monomers, such as polytetrafluoroethylene (PTFE) particles; and (c) a polysaccharide, such as chitosan,

In one embodiment, R₁ and R₂ are independently hydrocarbon groups from one to 12 carbons (typically from 1 to 3). In a preferred embodiment according to the invention, R₁ is —CH₂—, R₂ is —(CH₂CH₂CH₂)—, and R₃ is methyl or ethyl at each occurrence.

The R₃ groups may be the same group, or may be different groups at each occurrence. For example, two R₃ groups may be the same, all three R₃ groups may he different (e.g., mixed alkyl), or all three R₃ groups may be the same.

In a particular embodiment, the epoxy-functionalized inorganic oxide particles are silica particles functionalized with at least one epoxysilane having the structure:

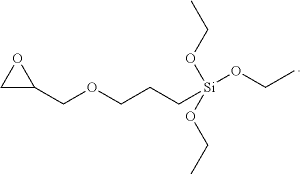 [SMILES: CCO[Si](CCCOCC1CO1)(OCC)OCC]

In a particular embodiment, methods are provided for holding a keratin fiber in a desired shape or style, comprising:
applying a sol-gel composition to a keratin fiber;
said sol-gel composition comprising a dispersion of (a) epoxy-functionalized inorganic oxide particles; (b) a cosmetic particulate selected from the group consisting of inorganic particulates, organic polymeric particulates, pigments, lakes, silicon-based particulates, and combinations thereof; and (c) chitosan or a chitosan derivative;
imparting a desired shape or style to said keratin fiber; and allowing said keratin fiber with said sol-gel composition thereon to dry.

In a particular embodiment, methods are encompassed for holding hair to a desired shape or style, comprising applying a sol-gel composition to hair in need thereof, wherein the sol-gel composition comprises a dispersion of (a) silica particles functionalized with an epoxysilane having the structure:

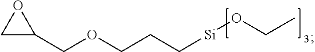

(b) polytetrafluoroethylene (PIPE) particles; and (c) chitosan or a chitosan derivative, applying said sol-gel composition to said hair, shaping and/or styling said hair to a desired shape, and allowing said sol-gel composition to dry on said hair without application of heat, i.e., without applying external heat, or at a temperature of about 73° F. (about 23° C.). In other embodiments, the sol-gel composition is dried on the hair at a temperature less than about 200° F. (about 93° C.), or less than about 150° F. (about 66° C.), or less than about 100° F. (about 38° C.).

In one embodiment of the invention, methods are provided for providing a durable film on a human integument, comprising:
applying a sol-gel composition to a human integument, wherein said sol-gel composition comprises a dispersion of:
(a) epoxy-functionalized inorganic oxide particles;
(b) a cosmetic particulate selected from the group consisting of inorganic particulates, organic polymeric particulates, pigments, lakes, silicon-based particulates, and combinations thereof; and
(c) an amino-functionalized polysaccharide; and drying said human integument with said sol-gel composition thereon to form a durable film on said human integument.

Epoxysilanes of particular interest for frictionalizing silica particles to provide epoxysilane-functionalized silica particles according to the invention include glycidyloxypropyl groups such as 3-glycidoxypropyltrimethoxysilane (GLYEO), 3-glycidoxypropyltriethoxysilane and 3-glycidoxypropyl-methyldiethoxysilane. Of particular interest according to the invention are mono-, di-, or tri-functional silyl groups,

In other embodiments, the silica particles may be treated with an alkoxysilane, alone or in combination with the expoxysilanes described above, Appropriate alkoxysilyl residues for functionalizing silica particles are, for example, silanes selected from the group comprising 3-aminopropyltriethoxysilane, aminoethylaminopropyl trimethoxysilane, aminoethytamino-propyitrimethoxysilane, aminoethytaminopropylsilane, 3-aminopropyltrimethoxysilane, N-(2-aminoethyl)-3-aminopropyltrimethoxysitane, N-(2-aminoethyl)-3-aminopropyl-methyldimethoxysilane, N-(2-aminoethyl)-3-aminopropylmethyl-dimethoxysilane, N-cyclohexyl-3-aminopropyltrimethoxysilane, benzytarninoethylatninopropyitrimethoxy-silane, vinyihenzylamino-ethylaminopropyltrimethoxysilane, vinyltrimethoxysilane, vinyltriethoxysilane, vinyklimethoxymethylsilane, vinyhtris)methoxyethoxy)silane, vinylmethoxymethylsilane, vinyl-tris(2-methoxyethoxy)silane, vinyttriacetoxysilane, methyltrimethoxysilane, n-hexyltrimethoxysilane, n-octyltriethoxysilane, propyltrimethoxysilane, propyltriethoxysilane, t-butyltrimethoxysilane, isohutyltriethoxysilane, ehloropropyltrimethoxysilane, 3-glycidoxypropyltrimethoxysilane, 3-glycidoxypropyltriethoxysilane, glycidoxy-propylmethyldiethoxysilane, phenytrimethoxysilane, phenyttriethoxysilane, mercaptopropyltrimethoxysilane, bis-triethoxysitylpropyl disulfide silane, bis-triethoxysilylpropyl disulfide same, bis-triethoxysilyi-propyltetroasuilidosilane, tetraethoxysilane, N-cyclohexylaminomethyldiethoxysilane, N-cyclohexytaminomethyltriethoxysilane, N-phenylaminomethyltrimethoxysilane, (Methacryloxymethyl)methyldimethoxysilane, methacrytoxymethyltrimethoxysilane, (methacryloxymethyl)-methyldiethoxysilane, methacryloxymethyltriethoxysilane, 3-methacryloxypropyl-trimethoxysilane, 3-methaciyioxypropyltrimethoxysiiane, 3-methacryloxypropyl-triacetoxysilane, (isocyanatomethyDmethyldimethoxysitane, 3-isocyanato-propyl-trimethoxysilane, 3-trimethoxysilylmethyl-O-methyl-carbamate, N-dimethoxy-(methyl)-silylmethyl-O-methyl-carbamate, 3-(triethoxysilyl)propyl succinic acid anhydride, methyltrimethoxysilane, methyltriethoxysilane, dimethyldimethoxysilane, dimethyldiethoxysilane, trimethytethoxysilane, isooctyltrimethoxysilane, isooctyl-triethoxysilane, hexadecyltrimethoxysilane, (octadecyl)methyidimethoxysilane, phenyl-triethoxysilane, (cyclohexyl)methydimethoxysilane, dicyclopentyldimethoxy-silane, and tetraethyl silicate. Mixtures of these compounds are also contemplated to be appropriate for use in the invention.

The epoxysilane-functionalized silica particles for use in the invention are particularly suitable when the epoxysilane-furtctionalized silica particles contain 0.5 to 10 moles, preferably 1 to 5 moles, epoxy groups per kg of epoxy-containing polymer particles.

Another component comprises a cosmetic particulate. In one embodiment, the cosmetic particulate comprises polymeric fluorocarbon particles, such as polytetrafluoroethylene (PIPE), which may be represented by the general structure —[C₂F₄]ₓ—. Other suitable cosmetic particulates include, but are not limited, to spherical or substantially spherical polymeric particles of polyethylene, polypropylene, polyvinylchloride (PVC) polyvinylidenefluoride (PVDF), perfluoroalkoxy polymer (PEA), polytrifluoroethylene, poly-trifluorochloroethylene, polyamide imide, polymethylmethacrylate (PMMA), polyetheretherketone (PEEK), polyethylene terephthalate polyester (PETP), polystyrene, polydimethylsiloxanes, polymethylsisesquioxane, polyarnidk. (Nylon) powder, Vinyl Dimethicone/Methicone Silsesquioxank. Crosspolymer, Polysilicones, and silicone elastomers, to name a few. Other useful powders include fatty acid derivatives of lysine, notably N-lauroyl lysine (including L-lysine and D-lysine). In some embodiments, the cosmetic particulate is a non-film forming cosmetic particulate, where “non-film forming” means that the cosmetic particulate does not form a continuous film on a surface after drying.

A particularly preferred cosmetic particulate according to the invention is PTFE.

The cosmetic particulate may be a single cosmetic particulate, or may be a mixture of more than one cosmetic particulate. A preferred cosmetic particulate is a non-dispersible polymeric particle and an insoluble polymeric particle.

The amount of the cosmetic particulate may be selected according to the intended use by the formulator, but is typically from about 1 to about 50 weight percent, preferably from about 1 to about 25 weight percent, based on the total weight of the sol-gel composition.

Another component comprises a biopolymer or polysaccharide, or mixtures thereof. In a specific embodiment, the biopolymer is chitosan or a chitosan derivative. A suitable chitosan is available commercially from C.E. Roeper. Of particular interest according to the invention is deacetylated chitosan, which may he at least about 40% deacetylated. Salts or derivatives of polysaccharides or chitosan may also be utilized in the compositions and methods of the invention. “Derivatives” may be obtained from biopolymers modified by chemical reaction(s), i.e., alkylated or acylated or otherwise modified by methods known in the art. Other suitable chitosans may be anionic, cationic, non-ionic, carboxylated, succinylated, alkoxylated, or quaternary chitosan derivatives, or carboxymethyl chitin (chitosan), chitosan salts, nano-chitosans, xylans, xylan derivatives, propolis, deacetylated derivatives, and mixtures thereof.

The polysaccharide or biopolymer (e.g., an amine-functionatized polysaccharide such as a mucopolysaccharide) optionally comprises at least one reactive functional group, wherein the reactive functional group is at least one group selected from amino (—NH₂), hydroxyl (—OH), carboxyl, —OR, —R—X (wherein X is a leaving group such as a halogen), —R—SO₃, and —R—SH groups, wherein R is a C₁-C₆ hydrocarbon (typically C₁-C₆ alkyl).

In some embodiments, the biopolymer or polysaccharide may be optionally functionalized by way of conversion with an organic side-chain residue of an expoxysilane, alkylsilyl, and/or siloxane compound(s).

The compositions may further comprise a shine enhancer, typically in an amount from 0.05 to 25% by weight of the total composition, to combat any matte finish. In various embodiments, the shine enhancer is, without limitation, a hemi-spherical methyl methacrylate crosspolymer, or an aryl silicone having a refractive index at 25° C. of greater than 1.4. Phenyltrimethicone is a non-limiting example of a typical aryl silicone.

The compositions may further comprise at least one film former, preferably a hydrophobic material, and is typically in an amount from 0.05 to 25% by weight of the total composition. The hydrophobic film former may be any hydrophobic film former suitable for use in a cosmetic composition, including, but not limited to, hydrophobic film-forming polymers.

In a related embodiment, a composition for shaping a keratin fiber is provided comprising about 85% by weight of the inventive aqueous sol-gel composition, about 10% by weight VP/DMAPA acrylates copolymer, and about 5% by weight cationic polymer (e.g., polyquaternium-37).

In another aspect of the invention, a method for straightening hair and/or reducing frizz in humid conditions and/or protecting the color of artificially colored hair is provided comprising applying to hair in need thereof any of the inventive compositions and allowing the solvents present to evaporate. The composition may be applied to wet hair or dry hair.

These and other aspects of the present invention will become apparent to those skilled in the art after a reading of the following detailed description of the invention, including the figures and appended claims.

### BRIEF DESCRIPTION OF THE DRAWINGS

FIG. 1 shows SEM/EDAX analysis of treated and untreated hair tresses according to the invention.

### DETAILED DESCRIPTION

All references to median or mean particle sizes herein are on a volume basis. All amounts provided in terms of weight percentage are relative to the entire composition unless otherwise stated.

As used herein, the term “non-dispersiblk.” when applied to the cosmetic particulate of the invention is intended to embrace particulates which are not solubilized, partially solubilized or dispersed in any quantity of solvent or solution. The particles for use in the instant invention are distinguished from “dispersible” particles, which are solubilized, partially solubilized and/or dispersed in some quantity of solvent or solution. “Non-film forming” as used herein is intended to embrace particles which when dried from a dispersion in a liquid do not form a continuous, adherent film on a substrate.

Unless otherwise provided, the terms “hydrocarbon” or “alkyl” are intended to embrace straight-chained, branched, or cyclic hydrocarbons, particularly those having from one to 20 carbon atoms, and more particularly C₁₋₁₂ alkyl, and more preferred still are C₁₋₆ alkyl groups. Methyl and ethyl are particularly preferred alkyl groups.

The term “human integument” as used herein in the methods of the invention includes, without limitation, keratinous fibers such as hair of the scalp, eyelashes, facial hair, eyebrows; skin, including lips, skin of the face, eyelids, neck, body, etc.; nails, and/or any other surface of a human body for which it may be desirable to impart a durable film. In certain embodiments, the human integument is selected from skin, nails, lips, eyelashes, and hair of scalp.

As used herein, the term “keratin fiber” includes hair of the scalp, eyelashes, eyebrows, acial hair, and body hair such as hair of the arms, legs, etc. In particular embodiments, the keratin fiber is hair of the scalp. Keratin fibers are not limited to humans and also include any keratin fibers from a mammal, such as, for example, pet hair and mammalian fur.

The inventive sol-gel compositions for application to keratin fibers will generally be silica sol-gel compositions, and will generally be aqueous, although emulsions, such as water-in-oil or oil-in-water emulsions are within the scope of the invention. As used herein, the water-in-oil emulsions include water-in-silicone emulsions. When reference is made to the weight percent of a component based on the weight of the total composition, the total weight of the composition will be understood to include both the aqueous and oil phases of the emulsion.

Acids may be included in the compositions, particularly to aid in solubilizing the polysaccharide component. “Acid” as used herein encompasses any water soluble or miscible acid, such as hydrochloric acid, nitric acid, sulfuric acid, or acetic acid. In a particular embodiment, the acid included is acetic acid.

The epoxy-functionalized silica particles may be formed from colloidal silica and epoxysilane, and will collectively comprise between about 1% to about 90% by weight of the final silica sol-gel composition, including water, and more typically will comprise from about 0.1% to about 10%, and preferably from about 0.5% to about 4%, and more preferred still, from about 1% to about 3% by weight of the composition.

The epoxy-functionatized silica particles according to the invention result in a sol-gel composition wherein the cosmetic particulate as well as the polysaccharide are homogeneously or evenly dispersed therein, wherein any given volume in such sol-gel compositions is chemically indistinguishable from any other volume of the sol-gel composition.

Examples of suitable epoxysilane-functionatized silica particles are commercially available, including, for example, products from the company Nano-X GmbH available under the name X-BOND® E. Solvent-reduced binders from the X-BOND® E series are preferred, in particular X-BOND® E 9013, X-BOND® E 9014, X-BOND® E 9017, X-BOND® E 9075, X-BOND® E 9076 and X-BOND® E 9077.

The epoxysilane-functionalized silica particles when combined with water for use in the invention may have a viscosity of from about 5 to about 2000 mPas, more preferably from about 10 to about 200 mPas (measured according to ISO 2555). The epoxysilane-functionalized silica particles when combined with water for use in the invention may have, for example, a solids content from about 1% to about 90% by weight, preferably from about 20% to about 60% by weight, based on the total weight of the combination.

The final weight ratios within the resulting inventive sol-gel composition according to the invention are controlled to produce compositions with the desired styling effects and aesthetics.

The cosmetic particulate will be homogenotisly distributed in the sol-gel composition. This cosmetic particulate may be present in weight percentages from about 0.01% to about 30%, preferably from about 2% to about 25%, and more preferred still, from about 5% to about 20% by weight of the composition,

This cosmetic particulate, such as PIPE, will typically have a median particle size between about 1 nm (nanometer) and about 1 micron, more typically between about 1 nm and about 300 nm, preferably between about 100 nm and about 300 nm, including 150 nm, 200 nm, 250 nm, or about 275 nm. In a preferred embodiments, the cosmetic particulate is PTFE, with a median particle size between from about 200 nm to about 10 micrometers, in particular of about 220 nm. Where the cosmetic particulates comprise inorganic particulates, including but not limited to TiO₂, BN, etc., the median particle size is preferably from about 40 nm to about 400 nm. Where the cosmetic particulate comprises pigments, iron oxide pigments will preferably have a median particle size between from about 300 nm to about 10 micrometers. Where the cosmetic particulate comprises organic particulates, the median particle size is preferably from about 200 nm to about 10 um. Where the cosmetic particulate comprises silicon-based particulates the median particle size is preferably from about 1 micrometer to about 20 micrometer. The cosmetic particulate may also comprise superfine/fumed silica with a median particle size from about 50 nm to about 400 nm. The cosmetic particulate may also comprise pearls with a median particle size up to about 100 microns.

Suitable, commercially available, cosmetic particulates include, without methylsilsesquioxane resin microspheres, for example, TOSPEARL™ 145A, (Toshiba Silicone); particles of polymethylsilsesquioxane sold under the name TOSPEARL™ 150 KA (Kobo); microspheres of polymethylmethacrylates, for example, MICROPEARL™ 100 (Seppic); spherical particles of polymethylmethacrylate, such as those sold under the name TECEEPOLYMER™ MB-8CA (KOBO)); particles of VinylDimethicorte/Methicone Silsesquioxane Crosspolymer sold under the name KSP™ 105 (Shin-Etsu); the spherical particles of crosslinked polydimethylsiloxanes, for example, TREFIL™ E 506C or TREHL™ E 505C (Dow Corning Toray Silicone); spherical particles of polyamide, for example, nylon-12, and ORGASOL™ 2002D Nat C05 (Atochem); polystyrene microspheres, for example Dyno Particles, sold under the name DYNOSPHERES™, and ethylene aciyiate copolymer, sold under the name FLOBEAD™ EA209 (Kobo); aluminum starch octenyisuccinate, for example DRY FLO™ (National Starch); microspheres of polyethylene, for example MKROTEIENE™ FN510-00 (Equistar), spherical particles of PTFE, available under the name FLUOROPURE™ 109 C (Shamrock) or MICROSLIP™ 519 (Presperse); silicone resin, polymethylsasesquioxane silicone polymer, Polysilicones, including without limitation, Polysilicone-1, Polysilcone-2, Polysilicone-3, Polysilicone-4, Polysilicone-5, Polysilicone-6, Polysilicone-7, Polysilicone-8, Polysilicone-10, Polysilicone-11, Polysilicone-12, Polysilicone-13, Polysilicone-14, Polysilicone-i15, Polysilicone-16, Polysilicone-17, Polysilicone-18, and Polysilicone-19; Dimethicone/Divinyidimethicone/Silsesquioxane Crosspolymer (available under the trade name GRANSIL EPSQ from Grant Industries); dimethicone/silsesquioxane copolymer (available under the trade name SILDERM EPSQ from Active Concepts); platelet shaped powder made from N-lauroyl lysine, available under the name AMIHOPE™ LL (Ajinomoto), and mixtures thereof, to name a few. Other suitable particulates include the particulate silicon wax sold under the trade name TEGOTOP™ 105 (Degussa/Goldschmidt Chemical Corporation) and the particulate vinyl polymer sold under the name MINCOR™ 300 (BASF).

Other suitable cosmetic particulates include polymeric fluorocarbons, such as polymeric fluorocarbon compounds available commercially, i.e., Hexafor 6240, Hexafor 6280 (Motion); Unidyne TG-5543, Unidyne TG 580, Unidyne TG 582 (Daikin); Anthydrin SC, Anthydrin NK (Zschimmer & Schwarz); Tubicoat RP 10 RT (CHT Beitlich); Dyneon PFA 6900 GZ, Dyneon PFA 6910 GZ, Dyneon TIF 5032 Z, Dyneon TF 5035 Z, and Dyneon TIF 5050 Z (Dyneon 3M).

Other suitable cosmetic particulates include organic or inorganic pigments or lakes. Exemplary organic pigments include, for example, FD&C dyes, D&C dyes, including D&C Red, Nos. 2, 5, 6, 7, 10, 11, 12, 13, 30 and 34, D&C Yellow No. 5, Blue No. 1, Violet No. 2, and the like. Exemplary inorganic pigments include, but are not limited to, CROMORHITIAL® Yellow, SUNFAST® Magenta, SUNFAST® Blue, metal oxides and metal hydroxides such as magnesium oxide, magnesium hydroxide, calcium. oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides, zinc oxides, barium oxide, composite oxides and composite hydroxides such as iron titanate, cobalt titanate and cobalt aluminate, and potassium ferricyanide (K₃Fe((N)₆), potassium ferrocyanide (K₄Fe(CN)₆), potassium ferrocyanide trihydrate (K₄Fe(CN)₆.3H₂O), ferric ammonium ferrocyanide, ultramarine blue, carbon black particles, and the like.

Other suitable cosmetic particulates include lakes, such as, for example, aluminum lakes (e.g., aluminum lakes of FD&C Yellow No. 5 and No. 6, aluminum lakes of FD&C No. 40, aluminum lakes of D&C Red Nos. 21, 22, 27, and 28, aluminum lakes of FD&C Blue No. 1, aluminum lakes of D&C Orange No. 5, and aluminum lakes of D&C Yellow No. 10), strontium lakes (e.g., strontium lakes of D&C Red No. 13), barium lakes (e.g., barium lakes of D&C Red No. 12), calcium lakes (e.g., calcium lakes of D&C Red Nos. 7, 11, 31 and 34), zirconium lakes (e.g., the zirconium lake of D&C Red No. 33), Green 3 Lake, Ext. Yellow 7 Lake, Orange 4 Lake, Red 28 Lake, and the like,

The cosmetic particulate may also be based on inorganic fillers such as talc, mica, silica, and mixtures thereof, or any of the clays disclosed in EP 1 640 419, the disclosure of which is hereby incorporated by reference. Other suitable cosmetic particulates include calcium carbonate, calcium sulfate, kaolin, magnesium carbonate, carmine, barium sulfate, mica, bismuth oxychloride, zinc stearate, manganese violet, bismuth citrate, hydroxyapatite, and zirconium silicate.

In one embodiment, cosmetic particulates may be suthice modified, with, for example, fluoropolymers, to adjust one or more characteristics of the coloring agent as described in, for example, U.S. Pat. Nos. 6,471,950, 5,482,547, and 4,832,944, the contents of which are hereby incorporated by reference. Fluoropolymers may be incorporated into the present disclosure as a coating on pigment particles that at least partially covers the surface of the pigment particles. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference.

Other suitable cosmetic particulate materials include polymer blends, polymer beads, polymer fibers, plates mixed with pigments, iron oxide coated beads and fibers and the like.

Chitosan, or other appropriate polysaccharide or biopolymer, may be present in weight percentages from about 0.01% to about 30%, preferably from about 0.1% to about 25%, and more preferred still, from about 0.2% to about 2% by weight of the composition. In one embodiment, chitosan is present from about 0.05 to about 5 weight percent.

The sol-gel composition comprises water or an aqueous solution, which may comprise from about 99% by weight to about 50% by weight, preferably about 90% to about 60%, and most preferably from about 90% to about 70% of the total composition. Alcohols, such as ethanol, are optionally present, up to about 40% by weight, preferably (if present) from about 0.1% by weight to about 25% by weight of the total composition.

In some embodiments, it may be desirable to include one or more agents that enhance the shine of hair treated with the compositions of the invention. The shine-enhancing agent is preferably hydrophobic. For example, tens-shaped particles such as hemi-spherical PMMA have been found suitable for imparting shine. One such commercially available material is a hemi-spherical methyl methacrylate crosspolymer sold under the trade name 3D TECH PW (Plain) XP (Kobo).

Silicone fluids, such as amyl-substituted siloxanes having high refractive indices are also useful as shine enhancers. Particular mention may be made of phenyltrimethicone, which is available under the trade names SCI-TEC PTM 100 (ISP) and PDM20 (Wacker-Belsil). The PDM20 material has a refractive index of 1.437 at 25° C. In general, any aryl-substituted silicone having a refractive index of greater than 1.4 at 25° C. is contemplated to be suitable for restoring shine to hair treated with the inventive compositions. Another suitable silicone fluid that enhances shine is amodimethicone.

The shine enhancer, if present, is typically present from about 0.01% to about 5% by weight of the composition (including solvent). More typically, the shine enhancer component will comprise from about 0.05% to about 2.5% by weight of the composition. Preferably, the shine enhancer will comprise from about 0.1% to about 1.5% by weight of the composition, including embodiments wherein the shine enhancer is present at about 0.1%, 0.3%, 0.5%, 0.75%, 1%, 1.25%, or 1.5% by weight of the composition.

In some embodiments, a film former may be included in the compositions according to the invention. Polymeric film formers include polyolefins, polyvinyls, polyacrylates, polyurethanes, silicones, polyamides, polyesters, fluoropolymers, polyethers, polyacetates, polycarbonates, polyimides, rubbers, epoxies, formaldehyde resins, and homopolymers and/or copolymers of the foregoing.

Suitable hydrophobic (lipophilic) film-forming polymers include, without limitation, those described in U.S. Pat. No. 7,037,515 to Kalafsky, et al.; U.S. Pat. No. 6,685,952 to Ma et al.; U.S. Pat. No. 6,464,969 to De La Poterie, et al.; U.S. Pat. No. 6,264,933 to Bodelin, et al.; U.S. Pat. No. 6,683,126 to Keller et al.; and U.S. Pat. No. 5,911,980 to Samour, et al., the disclosures of which are hereby incorporated by reference. Other film formers known in the art can be used advantageously in the composition.

Preferred polymeric film formers include acrylates, alkyl acrylates, polyurethanes, fluoropolymers such as FLUONTER™ (polyperfluoroperhydro-phenanthrene) and silicone polymers. Particularly preferred are silicone acrylates such as acryylates/dimethicone copolymers sold under the trade names KP-545 or KP-550 (Shin-Etsu Co.).

Other film formers that may be employed include, without limitation, natural, mineral and/or synthetic waxes. Natural waxes are those of animal origin, including without limitation beeswax, spermaceti, lanolin, and shellac wax, and those of vegetable origin, including without limitation camauba, candetilla, bayberry, and sugarcane wax, and the like. Mineral waxes contemplated to be useful include, without limitation ozokerite, ceresin, montan, paraffin, microcrystalline, petroleum, and petrolatum waxes. Synthetic waxes include, for example, Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX® Polyethylene (Baker Hughes Incorporated). Commercially available ethylene-α-olefin copolymer waxes include those sold under the tradename PETROLITE® Copolymers (Baker Hughes Incorporated). Another wax that is suitable is dimethiconol beeswax available from Noveon as ULTRABE™ dimethiconol ester. Combinations of any of the foregoing film formers are also contemplated to be suitable, including combinations or polymeric and non-polymeric film formers.

The collective weight of the film formers, if present, will typically be between about 0.1% and about 5% by weight, more typically between about 0.1% and about 2.5%, or between about 0.5% and about 1.5% by weight, based on the total weight of the composition.

In some embodiments, cationic polymers may be included as conditioning agents or thickeners, Suitable cationic polymers include, but are not limited to, Polyquaternium-4, Polyquaterniurn-5, Polyquaternium-6, Polyquatemium-7, Polyquaternium 10, Polyquaternium-22, Polyquaternium-37, Polyquaternium-47, or any combination thereof. Special mention may be made of Polyquatemium-37.

In some embodiments, the compositions are typically aqueous, but may also comprise a volatile solvent that is compatible with the methods of the invention. Volatile solvents may include volatile C₅₋₁₂ hydrocarbons, aromatic hydrocarbons (e.g., xylenes, toluene, etc.), ketones (e.g., actetone, methylethyl ketone, etc.), ethers (e.g., diethyl ether, methylethyl ether, etc.), perfluorohydrocarbons, hydrofluoroethers, Freons (HCFCs, CFCs), volatile silicones, lower alcohols (e.g., ethanol, propanol, isopropanol, butanol, etc.), esters of acetic acid (e.g., ethylacetate, butylacetate, etc.), and the like. Preferred volatile solvents will be cosmetically acceptable, by which is meant that they are safe and non.-irritating when applied to the body under conditions of normal use.

Volatile silicones are a preferred volatile solvent, By “volatile silicone” is meant that the fluid readily evaporates at ambient temperatures. Typically, volatile silicones will exhibit a vapor pressure ranging from about 1 Pa to about 2 kPa at 25° C.; will preferably have a viscosity of from about 0.1 to about 10 centistokes, preferably about 5 centistokes or less, more preferably about 2 centistokes or less, at 25° C.; and will boil at atmospheric pressure at from about 35° C. to about 250° C. Volatile silicones include cyclic and linear volatile dimethylsiloxane silicones, including 0.5 est dimethicone, 0.65 est dimethicone, 1 est dimethicone, and 1.5 est dimethicone. In one embodiment, the volatile silicones may include cyclodimethicones, including tetramer (D4), pentamer (D5), and hexamer (D6) cyclomethicones, or mixtures thereof. Suitable dimethicones are available from Dow Coming under the name DOW CORNING 200® Fluid and have viscosities ranging from 0.65 to 5 centistokes. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics,” Cosmetics and Toiletries, 91:27-32 (1976), herein incorporated by reference in its entirety. Linear volatile silicones generally have a viscosity of less than about 5 centistokes at 25° C. (77° F.), whereas the cyclic silicones have viscosities of less than about 10 centistokes at 25° C. (77° F.). Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SW-S-03314 (SWS Silicones Corp.), Linear, volatile silicones include low molecular weight polydimethylsitoxane compounds such as methyltrimethicone, trisiloxane, hexamethyldisiloxane, octamethyltrisiloxane, decamethyltetrasiloxane, and dodecamethylpentasiloxane to name a few.

Lower alcohol solvents, including methanol, ethanol, propanol, and isopropanol, are also contemplated to be useful. Ethanol is particularly preferred due to its high volatility and low toxicity.

Among the volatile C₅₋₁₂ hydrocarbons, special mention may be made of isododecane which is available under the trade name PER:METHYL-99A (Presperse Inc.), Suitable fluorinated solvents include, without limitation, perftuoroethers, perfluorodecalin, perfluoromethyidecalin, perfluorohexane, perfluorornethylcyclohexane, perfluorodimethylcyclohexarte, perfluoroheptane, pertluorooctane, perfluorononane, and perfluoromethylcycopentane, for example.

In addition to the foregoing, the compositions according to the invention may comprise additional pigments, pearlescents, and/or colorants to impart a desired color to the hair, provided that such components do not undesirably detract from the desired styling effect.

Various fillers and additional components may be added. Suitable fillers include, without limitation, silica, treated silica, talc, zinc stearate, mica, kaolin, Nylon powders such as ORGASOL™, polyethylene powder, TEFLON™, boron nitride, copolymer microspheres such as EXPANCEL™ (Nobel Industries), POLVIRAP™ (Dow Corning) and silicone resin microbeads (TOSPEARL™ from Toshiba), and the like. These fillers may be in addition to, or in place of, the second component particulate material.

The aggregate amount of such optional pigments, colorants, and fillers is not particularly restricted provided that the desired aesthetics of the treated hair and the methods of the invention are not compromised. Typically, the pigments, colorants, fillers, etc., if present, will collectively comprise from about 0.1% to about 25% of the composition (including volatile solvent), but more typically will comprise from about 0.1% to about 10%, or from about 0.5% to about 2.5% by weight of the composition.

The compositions of the invention may optionally comprise other active and inactive ingredients typically associated with cosmetic and personal care products, including, but not limited to, excipients, fillers, emulsifying agents, antioxidants, surfactants, film formers, chelating agents, gelling agents, thickeners, emollients, humectants, moisturizers, vitamins, minerals, viscosity and/or theology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skirt cooling compounds, skin protectants, skin penetration enhancers, exfollients, hibricants, fragrances, colorants, staining agents, depigmenting agents, hypopigmenting agents, preservatives, stabilizers, pharmaceutical agents, photostabilizing agents, and mixtures thereof. If present, the levels of such additional components preferably should be judiciously selected no as not to adversely impact the durability of the film. Collectively, all such additional components suitably will comprise less than 50% by weight of the composition, but will typically comprise less than about 20% by weight of the total composition.

In certain embodiments, the compositions according to the invention are free, or substantially free, of isocyanates and isocyanate containing compounds (including diisocyanates). By “substantially free of” it is meant that such isocyanate compounds comprise less than about 0.1%, preferably less than about 0.01%, and more preferred still less than about 0.001%, by weight of the composition. In any event, a composition substantially free of isocyanate will have an amount of isocyanate inadequate to impart measurable wear properties to the durable film.

The compositions of the invention in certain embodiments may be suitably prepared by first making a solution of a precursor (colloidal silica) in a solvent (e,g., water, and optionally other volatile solvents) and at least one epoxysilane, and producing epoxysilane-functionalized silica particles by condensation of the epoxysilane with the silica particles. The resulting epoxysilane-functionalized silica particles are then combined with the cosmetic particulate and the biopolymer material; and, if present, further combining the resulting silica. sol-gel with a shine enhancer and/or any other optional desired ingredients. There is essentially no restriction on the order of addition or manner of mixing these components. An acid such as acetic acid may be added.

In certain embodiments, the epoxysilane may be further optionally reacted with at least one reactive functional group on a polysaccharide, for example chitosan, instead of, or in addition to, reacting with at least one reactive functional group on colloidal silica.

The reactive functional groups of the polysaccharide may be any functional groups that are reactive with either the silane or the epoxy functional groups of the epoxysilane, including but not limited to amino (—NH₂), hydroxyl (—OH), carboxyl, —OR, —R—X (wherein X is a leaving group such as a halogen), —R—SH, and —R—SO₃ groups, wherein R is a C₁-C₆ hydrocarbon, in particular, a C₁-C₆ alkyl.

The composition may be mixed or homogenized at room temperature. Once complete, the composition can be packaged, for example into a pump, or a jar accessible with the fingers. The cosmetic compositions are ideally intended for pump or manual delivery to the hair. In one embodiment, a cosmetic product is provided comprising a packaging substantially impermeable to air, having disposed therein a charge of cosmetic composition comprising a silica sol gel according to the invention. The packaging may be molded from plastic or the like and may be equipped with a dispenser such as a pump for dispensing the silica sol-get without opening the container to the airAlternatively, the container may have an orifice, covered with a removable cap, through which the cosmetic product may be removed by the fingers or the like. In some embodiments, an aerosolizing agent is added; while in other embodiments, the formulation is a non-aerosol formulation.

In certain embodiments according to the invention, the percentage of the silane groups of the added epoxysilane which have reacted with the hydroxyl groups on the silica particles may be from about 0.1 to about 100%, from about 20 to about 100%, or from about 50 to about 100%.

In certain embodiments according to the invention, the percentage of the epoxide groups of the added epoxysilane which have optionally reacted with the amine groups on the chitosan may he from about 0 to about 100%, from about 0 to about 50%, from about 0 to about 10%, or from about 0 to about 1%. In some, but not all, embodiments, the amount of reacted. amine groups is at least 0.001 percent by weight of the epoxide groups of the added epoxysilane.

In certain embodiments according to the invention, the percentage of the silane groups of the added epoxysilane which have optionally reacted with the amine groups on the chitosan may be from about 0 to about 99.9%, from about 0 to about 50%, from about 0 to about 10%, or from about 0 to about 1%. In some, but not all, embodiments, the amount of reacted. amine groups is at least 0.001 percent by weight of the silane groups of the added epoxysilane. The silica sot-gel compositions according to the invention are intended for topical application to human integument, in certain embodiments for application to the hair (hair of the body, scalp, beard, mustache, eyelashes, etc.).

In certain embodiments, the silica sot-gel compositions according to the invention are utilized in methods to impart a desired shape or style to a keratin fiber, including but not limited methods of decreasing the frizziness of hair, smoothing frizzy hair, straightening curly hair, to preserve the color of artificially colored hair, and to improve the aesthetic appearance of hair. The foregoing may be tested using hair swatches treated with the inventive cosmetic compositions. Likewise, the compositions may be applied to the hair of a wig, to impart shape, decrease frizziness, etc. Likewise, the compositions may by utilized in, without limitation, skin care and/or color cosmetic compositions.

In certain embodiments, the inventive composition may be applied, preferably applied with the hands, onto dry or wet human integument, including, for example, dry hair or wet hair. It has surprisingly been found the aesthetic effects may be achieved even in the case where the wet hair is not dried with heat, or where the wet hair is dried with low heat. Additionally, it has been found that the effects of the inventive compositions are maintained after at least one, at least two, or even more, washings and/or shampooings.

In certain embodiments, the methods of the invention include drying of the inventive composition at room or ambient temperature without applying external heat, or at a temperature of about 73° F. (about 23° C.), or at a temperature less than about 200° F. (about 93° C.), or less than about 150° F. (about 66° C.), or less than about 100° F. (about 38° C.).

In certain embodiments, the methods and compositions of the invention include cosmetic and personal care applications, in particular, methods of application to a human integument.

Additional components may be incorporated into compositions of the invention as fillers or for various functional purposes as is customary in the art. However, while additional components consistent to formulate the above cosmetic compositions may be included, the inclusion of additional ingredients is limited to those ingredients which do not interfere with the aesthetic effects on the hair or other human integument. Additional components must also be compatible for use in cosmetic compositions for application to a human integument.

### EXAMPLES

The examples below illustrate the effects on shaping, effects of humidity, and feel of hair from application of various cosmetic compositions according to the invention.

### Example I

The effect of adding silica sol-gel compositions according to the invention to hair on straightening and anti-frizz aesthetics was investigated. The appearance, tactile feel and effect of humidity on hair sample tresses treated with a silica sol-gel composition according to the invention were examined. The epoxysitane-functionatized silica particles were formulated by combining, in an aqueous solution, colloidal silica (SiO₂), and an epoxysitane (gycidyloxypropoxytriethoxysilane) having the following structure:

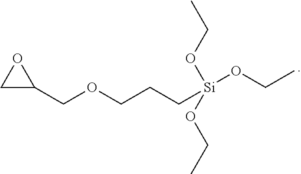 [SMILES: CCO[Si](CCCOCC1CO1)(OCC)OCC]

A condensation reaction produced epoxysilane-functionalized silica particles for use in the invention. The cosmetic particulate (PTFE particles) and the chitosan were added with stirring until a homogeneous sol-gel composition was obtained. Sufficient 5% aqueous acetic acid was added to solubilize the chitosan.The components may be added in any order.

The sample sol-gel composition had the following components by weight percent:

3.5% acetic acid;

0.6% alcohol (Ethanol);

0.35% chitosan (available from Biolog GmbH);

0.8% hydrated silica (Solum Diatomeae, Klebosol Ph 30HB25K, available from Univar GmbH, Essen);

11% 220 rim PTFE particles (Alkyl Phenol Ethoxylate; Dyneon TF 5035 Z, available from Dyneon GmbH, Burgkirchen);

1.5% binder (epoxysitane-functionalized silica particles); and

82.25% water.

This silica sol-gel was tested according to the following protocols.

### Hydrophobicity, Tactile Feel and Appearance Test Method

The silica sol-gel composition to be evaluated, or water as a negative control, was applied to European curly hair sample tresses measuring approximately ¾×8 inches (International Hair Importers, Glendale, N.Y.). The tress was wet with water and toweled dry. The silica sol-gel samples were applied to a damp tress with the fingertips and then combed through twice with a small toothed comb. The tresses were then blow dried for three minutes or until completely dry, followed by treatment with a flat iron (6 strokes, five seconds each). Treated tresses were then placed in a humidity chamber at 85% RH and about 27° C. (81° F.) for three hours. A visual and tactile examination was made hourly and compared with a negative (plain water) controls. It was observed that the silica sol-gel treated sample showed significantly decreased frizz compared to the water-treated negative control, having from one-third to half the volume of untreated hair.

In order to test the durability of the treatment, previously treated tresses were washed with 12% sodium laureth sulfate (SLES) and blow dried for 15 minutes on the low setting (Conair bonnet dryer) before repeating the three hour humidity chamber treatment.

A visual and tactile examination was made of the tresses hourly. The experiment was performed in duplicate. It was observed that the silica sol-gel sample retained decreased frizz even after washing, having from one-third to half the volume of untreated hair.

### Example II

The effect of adding the silica sol-gel composition according to the invention to hair on straightening and anti-frizz aesthetics are disclosed. The appearance, tactile feel and effect of humidity on hair sample tresses treated with the exemplary compositions, but without heat treatment, were examined.

Samples of the silica sol-gel composition according to the invention, or water as a negative control, were applied to European curly hair (International Hair Importers, Glendale, N.Y.). Approximately 0.25 g sample per gram of hair was massaged into the hair with fingertips and then combed through 3 times with a small tooth comb. The tresses were allowed to air dry at ambient temperature. Tresses were subsequently washed with 12% aqueous sodium lauryl ether sulfate (0.5 mL per tress) and combed through with a small tooth comb. Tresses were allowed to air dry again at ambient temperature. The experiment was performed in duplicate.

A visual and tactile examination was made of the dry sample tresses on the following day. The silica sol-gel sample was straight after this treatment, having approximately half the volume as the untreated sample. The tresses felt soft and seemed to have little residue. The results showed that heat was not required for straightening tresses with the silica sot-gel composition, as the silica sol-gel composition treated tresses retailed their straightness and smoothness compared to the water treated control sample.

### Example III

The treated tresses were examined by Scanning Electron Microscopy and Energy Dispersive Spectroscopy (SEM EDAX) to determine the durability of the compositions when applied to hair, i.e., the extent to which the inventive compositions were retained as a film on the Latina curly hair sample tresses. The silica sol-gel composition and water control samples were applied to Latin curly hair tresses (International Hair Importers, Glendale, N.Y.) by applying 0.25 g sample per gram of hair to the tresses. The sample was massaged into the hair with the fingertips and combed through 2 times with the small tooth side of a comb. The tresses were blow dried for 3 minutes (or until completely dry), then fiat ironed at 200° C. (392° F.) for 6 strokes (5 seconds per stroke). A few strands of hair were set aside for SEM/EDAX analysis. The tresses were then subsequently washed with 12% aqueous sodium lauryl ether sulfate (0.5 mL per tress). Tresses were dried with a Conair bonnet dryer for 15 minutes on low. A few strands of hair were set aside for SEM/EDAX analysis.

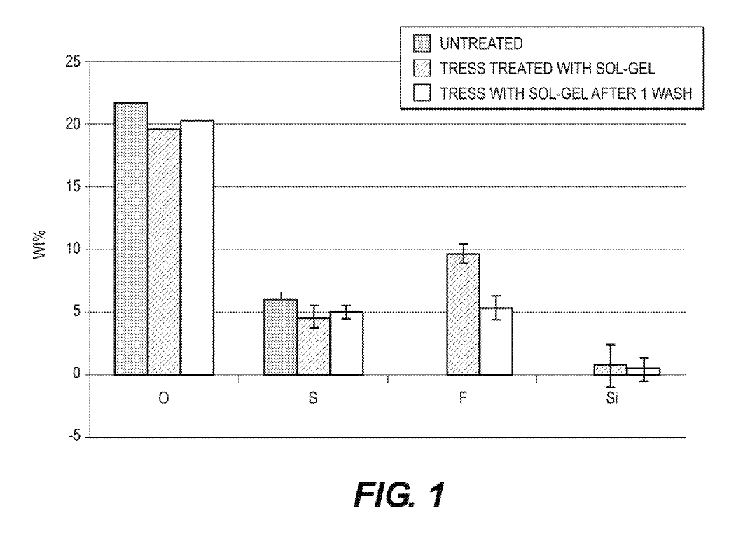

The silica sol-gel composition treated tress was tested, and an untreated tress was used as the reference. Scanning electron microscopy (SEM) coupled with energy-dispersive X-ray spectroscopy (EDAX) gave the relative weight % of each element of interest, i,e., oxygen (O), sulfur (S), fluorine (F), and silicon (Si). As seen in FIG. 1, the inventive silica sol-gel composition was retained on the samples tresses as shown by the fluorine and silica weight percentage compared to the untreated tress after one wash with 12% aqueous sodium lauryi ether sulfate.

### Example IV

The individual components of the silica sol-gel composition, together and separately, were tested according to the protocol of Example I. Epoxysilane-functionalized silica particles, water, PTFE cosmetic particulate, or chitosan, when applied alone to sample tresses, did not provide the straightening and smoothing effect of the silica sol-gel composition sample. Similarly, a combination of only the epoxysitane-functionalized silica particles and PTFE cosmetic particulate did not result in the straightening and smoothing effect provided by the silica sol-gel composition having a homogeneous dispersion of epoxysilane-functionalized silica particles, PTFE cosmetic particulate, and chitosan. These results indicate that all components are necessary in the silica sol-gel composition to get the desired straightening and aesthetic benefits.

### Example V

A sample silica sol-gel containing formula, suitable for application to human hair, illustrating approximate weight percentages is given in Table 1.

TABLE 1

| Ingredient | Weight % |
|---|---|
| Silica sol-gel | 85 |
| VP/DMAPA Acrylates Copolymer | 10 |
| Polyquaternium-37 | 5 |

### Example VI

A sample silica sol-gel containing formula, suitable for application to human hair, illustrating approximate weight percentages is given in Table 2.

TABLE 2

| Ingredient | Weight % |
|---|---|
| Silica sol-gel | 68 |
| Water | 12 |
| VP/DMAPA Acrylates Copolymer | 10 |
| Polyquaternium-37 | 5 |
| Dimethicone/dimethiconol | 5 |

### Example VII

A sample silica sol-get containing formula according to the invention may he formulated as follows (all values are approximate).

TABLE 3

| Component Name - INCI | | Exemplary |
|---|---|---|
| Nomenclature | Weight % | Range % |
| Ethyl alcohol | 0.56 | 0.05-2.5 |
| Chitosan | 0.32 | 0.05-2.5 |
| Acetic acid | 3.42 | 0.05-10 |
| Hydrated silica | 0.25 | 0.05-2.5 |
| PTFE (emulsified) | 6.85% | 0.5-2.5 |
| binder (epoxysilane- | 0.6 | 0.05-2.5 |
| functionalized silica particles) | | |
| Water | 88 | 50-99 |

### Example VIII

The effect of the sol-gel compositions according to the invention on artificially colored hair will be accomplished according to the Hydrophobicity, Tactile Feel and Appearance Test Method described, or by other tests known to those in the art.

The invention described and claimed herein is not to be limited in scope by the specific embodiments herein disclosed since these embodiments are intended as illustrations of several aspects of the invention. Any equivalent embodiments are intended to be within the scope of this invention indeed, various modifications of the invention in addition to those shown and described herein will become apparent to those skilled in the art from the foregoing description, Examples, and figure. Such modifications are also intended to fall within the scope of the appended claims. All publications cited herein are incorporated by reference in their entirety.

1. A method for holding a keratin fiber in a desired shape or style, comprising:
applying a sol-gel composition to a keratin fiber;
said sol-gel composition comprising an aqueous dispersion of (a) epoxy-functionalized inorganic oxide particles; (b) a cosmetic particulate selected from the group consisting of inorganic particulates, organic polymeric particulates, pigments, lakes, silicon-based particulates, and combinations thereof; and (c) chitosan or a chitosan derivative;
imparting a desired shape or style to said keratin fiber; and
allowing said sol-gel composition to dry.

2. The method according to claim 1, wherein said epoxy-functionalized inorganic oxide particles comprise epoxysilane functionalized silica particles.

3. The method according to claim 2, wherein said cosmetic particulate comprises organic polymeric particulates formed from polymerization of fluorinated olefin monomers.

4. The method according to claim 3, wherein said organic polymeric particulates comprise polytetrafluoroethylene (PTFE).

5. The method according to claim 4, wherein said keratin fiber is hair of the scalp.

6. The method according to claim 5, where said desired shape or style is smoothing frizzy hair and/or straightening curly hair,

7. The method according to claim 1, wherein said desired shape or style is maintained after at least one washing and/or shampooing.

8. The method according to claim 1, wherein said sol-gel composition comprises an aqueous dispersion of (a) silica particles functionalized with an epoxysilane having the structure: 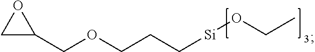
(b) polytetrafluoroethylene (PTFE) particles; and (c) chitosan or a chitosan derivative, and allowing said treated keratin fiber to dry without application of heat.

9. The method according to claim 8, wherein said desired shape or style is maintained after at least one washing and/or shampooing.

10. A method for providing a durable film on a human integument, comprising:
applying a sol-gel composition to a human integument, wherein said sol-gel composition comprises an aqueous dispersion of:
(a) epoxy-functionalized inorganic oxide particles;
(b) a cosmetic particulate selected from the group consisting of inorganic particulates, organic polymeric particulates, pigments, lakes, silicon-based particulates, and combinations thereof; and
(c) an amino-functionalized polysaccharide; and
drying said sol-gel composition to form a durable film on said human integument.

11. The method according to claim 10, wherein said drying is performed at ambient temperature.

12. The method according to claim 10, wherein said drying is performed at a temperature of less than about 200° F.

13. The method according to claim 12, wherein said drying is performed at a temperature of less than about 150° F.

14. The method according to claim 13, wherein said drying is performed at a temperature of less than about 100° F.

15. The method according to claim 10, wherein said (a) epoxy-functionalized inorganic oxide particles are silica particles functionalized with an epoxysilane having the structure: 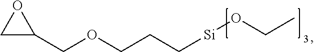
wherein said epoxy-functionalized inorganic oxide particles are from about 1 to about 3 weight percent;
said (b) cosmetic particulate is polytetrafluoroethylene (PTFE) with an average particle size from about 100 to about 300 nm and is from about 5 to about 20 weight percent; and
said (c) chitosan or chitosan derivative is from about 0.05 to about 5 weight percent.

16. The method according to claim 10, wherein said human integument is selected from skin, nails, tips, eyelashes, and hair of scalp.

17. The method according to claim 16, wherein said human integument is hair of the scalp, and further comprising applying a desired shape or style to said hair of the scalp.
